(19) United States
(12) Patent Application Publication
(10) Pub. No.: US 2011/0039282 A1
(43) Pub. Date: Feb. 17, 2011

(54) METHOD OF DETECTING CALCIFYING NANOPARTICLES AND SUSCEPTIBILITY TO CALCIFYING NANOPARTICLE INFORMATION

(75) Inventors: James X. Hartman, Boca Raton, FL (US); Patricia Keating, Boca Raton, FL (US)

Correspondence Address: Gregory A. Nelson;Novak Druce & Quigg LLP, 525 Okeechobee Blvd, Suite 1500, West Palm Beach, FL, 33401, US

(73) Assignee: Florida Atlantic University, Boca Raton, FL (US)

(21) Appl. No.: 12/988,201

(22) PCT Filed: Apr. 21, 2009
(86) PCT No.: PCT/US2009/041183

§ 371 Date: Oct. 15, 2010

Related U.S. Application Data
(60) Provisional application No. 61/046,651, filed on Apr. 21, 2008.

(51) Int. Cl. G01N 33/53; C12Q 1/02

(52) U.S. Cl. 435/7.92; 435/29

(57) ABSTRACT

A method of measuring the susceptibility of a subject to calcifying nanoparticle formation is disclosed. The method can include obtaining a cell culture comprising peripheral blood mononuclear cells, immune cells, or both, from a peripheral blood mononuclear fraction of a subject in a complete culture media; extracting a first aliquot and a second aliquot from the cell culture; and dispensing the first and second aliquot in different wells. The surface of the well containing the first aliquot can include a calcifying nanoparticle biofilm, while the well containing the second aliquot does not. The first and second aliquots can be incubated and the concentration of osteopontin in each aliquot determined. An osteopontin factor can be calculated and compared to osteopontin factors from a representative sample of other subjects. The osteopontin factor can be defined as the osteopontin concentration in the first aliquot divided by the osteopontin concentration in the second aliquot.

### TECHNICAL FIELD

The invention relates to a method of detecting calcifying nanoparticles in a fluid and susceptibility to calcifying nanoparticle formation using osteopontin.

### BACKGROUND

Although many facets of the immune response to most agents of disease have been heavily studied, there are no reports on the interaction of calcifying nanoparticles (CNPs), previously referred to as nanobacteria and nanons, with immune cells. It is believed that calcifying nanoparticles may be associated with numerous pathologies involving the presence of soft tissue calcification, such as atherosclerosis, rheumatoid arthritis, polycystic kidney disease, kidney stones, gallstones, prostatitis, and ovarian and breast cancer.

CNPs have been identified using a mouse monoclonal antibody, 8D10, by immune-histochemical methods and ELISA. Several groups have utilized the 8D10 antibody for the detection of CNPs in atherosclerotic plaque and aneurysms, psamomma bodies, and other calcifying disorders.

### SUMMARY OF THE INVENTION

The present invention is directed to a method of measuring the susceptibility of a subject to calcifying nanoparticle formation that includes, producing a cell culture comprising a complete culture media and peripheral blood mononuclear cells, immune cells, or both, obtained from a peripheral blood mononuclear fraction of a subject. A first aliquot and a second aliquot can be extracted from the peripheral blood mononuclear cell culture. The first aliquot can be dispensed in a first well and the second aliquot in a second well. The surface of the first well can have a calcifying nanoparticle biofilm, whereas the surface of the second well can be free of calcifying nanoparticles. The first and second aliquots can then be incubated and the concentration of osteopontin in the first and second aliquots determined. The osteopontin factor of the subject can be calculated by dividing the concentration of osteopontin in the first aliquot by the concentration of osteopontin in the second aliquot. The subject's osteopontin factor can then be compared to osteopontin factors from other individuals with varying levels of susceptibility to calcifying nanoparticle formation. The subject's susceptibility can be determined by correlating the osteopontin factor to the susceptibility to calcified nanoparticle formation based on the osteopontin factors from other individuals with varying levels of susceptibility of calcifying nanoparticle formation.

The cell culture can include peripheral blood mononuclear cells from a peripheral blood mononuclear fraction. The cell culture can include immune cells from a peripheral blood mononuclear fraction.

The culture media can be a human blood cell culture that includes 5-20% fetal bovine serum or media that is devoid of fetal bovine serum. The culture media can be free of any viable nanoparticles capable of replicating and can be chemically defined.

The subject can be identified as being susceptible to calcifying nanoparticle formation if the osteopontin factor is greater than about 5. The subject can be identified as being susceptible to calcifying nanoparticle formation if the osteopontin factor is greater than about 10.

The invention is also drawn to a method of measuring the susceptibility of a subject to calcifying nanoparticle formation that includes determining the concentration of osteopontin in a sample containing serum or de-fibrinated plasma from a subject. The concentration of osteopontin in the sample can be compared to the average osteopontin concentration of a population of subjects with a known resistance to calcifying nanoparticle formation.

The population of subjects with a known resistance to calcifying nanoparticle formation can be subjects who do not show signs of a calcifying nanoparticle formation. The susceptibility of a subject to calcifying nanoparticle formation is elevated if the osteopontin concentration of the subject is equal to or less than 50% of the average osteopontin concentration in a population of subjects. The concentration of osteopontin can be determined using ELISA or other immunological and analytical means routinely used for assay of proteins in biological fluids.

The invention can also be drawn to a method of diagnosing the presence of on-going calcifying nanoparticle formation that includes determining the concentration of osteopontin in a sample containing serum or de-fibrinated plasma from a subject. The osteopontin concentration of the subject can be compared to an average osteopontin concentration of a population of subjects with a known resistance to calcifying nanoparticle formation.

The invention is also drawn to a method of detecting the presence of calcified nanoparticles in a biological fluid that include separating serous fluid from biological solids in a biological fluid. A first aliquot that includes the serous fluid and cells from a cell line can be made and a second aliquot that includes a complete culture media and cells from the cell line can be made. The first aliquot can be dispensed in a first well and the second aliquot in a second well. The first and second aliquots can be incubated and then the concentration of osteopontin in each of the first aliquot and second aliquots can be determined. An osteopontin factor of the subject can be calculated by dividing the concentration of osteopontin in the first aliquot by the concentration of osteopontin in the second aliquot. The method can indicate the presence of calcified nanoparticles in the biological fluid if the osteopontin factor is greater than about 5.

The cell line can be a monocyte or macrophage derived cell line. The cell line can be a monocyte cell line selected from the group consisting of a THP cell line and a Monocyte/Macrophage cell line. The cell line can be any suitable monocytic cell line, preferably a cell line that is established and readily available.

The concentration of cells from the cell line in the first aliquot can be about the same as the concentration of cells from the cell line in the second aliquot. The concentration of cells from the cell line in the first aliquot can be the same as the concentration of cells from the cell line in the second aliquot. As used herein, “about the same” concentration refers to a difference in concentrations that is less than 15%, less than 5%, less than 2.5% or even less than 1%.

Prior to the addition of the first and second aliquots, the first well and the second well can be free of calcifying nanoparticles. The method can indicate the presence of calcified nanoparticles in the biological fluid if the osteopontin factor is greater than about 10.

These and other embodiments are described in more detail below.

### BRIEF DESCRIPTION OF THE DRAWINGS

The invention is described with more particularity below. The above and further advantages of this invention may be better understood by referring to the following descriptions taken in conjunction with the accompanying figures, in which:

FIG. 1 is a graph showing the secretion of osteopontin by PBMCs of individuals with systemic lupus erythrematosus (SLE) when the PBMCs are stimulated with CNPs or media alone.

FIG. 2 is a graph showing the secretion of osteopontin by healthy donor PBMCs from three healthy donors.

### DETAILED DESCRIPTION

Methods described herein can be used to detect the susceptibility of a subject to calcifying nanoparticle (CNP) formation. The methods can be used to detect the presence of an existing CNP formation. Heretofore, no one has been able to correlate immune resistance to or presence of a CNP formation to a marker present in bodily fluids. It has unexpectedly been discovered that the osteopontin level in bodily fluids can be used to detect the susceptibility of a subject to calcifying nanoparticle (CNP) formation and detect the presence of an on-going CNP formation. As used herein, the phrase “susceptibility of a subject” is used to indicate the likelihood that the subject will be affected with a disease, formation or condition.

As used herein, “CNP formation” is used to refer to the presence of an elevated quantity of individual CNPs or the formation of agglomerations or aggregates of a plurality of CNPs, which can cause calcification of tissue or calcified growths. For example, CNP formation can be used to describe ectopic calcifications, such as those that can occur in arteries, joints, and other soft tissues, and those that are formed as the result of diseases, including cancers, such as breast cancer and prostate cancer.

The present invention includes a method of measuring the susceptibility of a subject to calcifying nanoparticle formation that includes, producing a cell culture comprising a complete culture media and peripheral blood mononuclear cells, immune cells, or both, obtained from a peripheral blood mononuclear fraction of a subject. A first aliquot and a second aliquot can be extracted from the peripheral blood mononuclear cell culture. The first aliquot can be dispensed in a first well and the second aliquot in a second well. The surface of the first well can have a calcifying nanoparticle biofilm, whereas the surface of the second well can be free of calcifying nanoparticles. The first and second aliquots can then be incubated and the concentration of osteopontin in the first and second aliquots determined. The subject's osteopontin factor can be calculated by dividing the concentration of osteopontin in the first aliquot by the concentration of osteopontin in the second aliquot. The subject's osteopontin factor can then be compared to osteopontin factors from other individuals with varying levels of susceptibility to calcifying nanoparticle formation.

The cell culture can include peripheral blood mononuclear cells from a peripheral blood mononuclear fraction. The cell culture can include immune cells from a peripheral blood mononuclear fraction.

The culture media can be a human blood cell culture that includes 5-20% fetal bovine serum or media that is devoid of fetal bovine serum. The culture media can be free of any viable nanoparticles capable of replicating and be chemically defined. The culture media can be serum free.

As used herein, a chemically defined media is any media in which all experimentally relevant components and their respective concentrations are known. Having complete knowledge of the experimentally relevant components prevents the culture media from providing false or inaccurate results. For, example, fetal bovine serum may include components that can obscure results. Thus, the culture media described herein can be serum free media that is chemically defined with a constant and repeatable concentration of all experimentally relevant components.

The subject can be identified as being susceptible to calcifying nanoparticle formation if the osteopontin factor is greater than about 5. The subject can be identified as being susceptible to calcifying nanoparticle formation if the osteopontin factor is greater than about 10.

The invention is also drawn to a method of measuring the susceptibility of a subject to calcifying nanoparticle formation that includes determining the concentration of osteopontin in a sample comprising serum or defibrinated plasma from a subject. The concentration of osteopontin in the sample can be compared to the average osteopontin concentration of a population of subjects with a known resistance to calcifying nanoparticle formation.

The population of subjects with a known resistance to calcifying nanoparticle formation can be subjects who do not show signs of a calcifying nanoparticle formation. The susceptibility of a subject to calcifying nanoparticle formation is elevated if the osteopontin concentration of the subject is equal to or less than about 50% of the average osteopontin concentration of a population of subjects. The concentration of osteopontin can be determined using ELISA or other methods based upon the use of a monoclonal antibody specific for osteopontin including, but not limited to, western blot and lateral immunodiffusion. Additional methods for determining the level of osteopontin include, but are not limited to, chromatographically based instrumentation such as high pressure liquid chromatography.

The invention is also drawn to a method of diagnosing the presence of an existing calcifying nanoparticle formation that includes determining the concentration of osteopontin in a sample comprising serum or defibrinated plasma from a subject. The osteopontin concentration of the subject can be compared to an average osteopontin concentration of a population of subjects with a known resistance to calcifying nanoparticle formation.

For example, the population of subjects can be developed by taking serum or defibrinated plasma samples from 100 men and 100 women can be taken and the osteopontin concentration determined. The average and standard deviation can be calculated for men, women, and the entire population. Outliers may be eliminated prior to calculating the average and the standard deviation. Although this example is provided, it would be understood that the method of determining an appropriate average may be determined using any number of statistical, probabilistic, or other techniques.

The concentration of osteopontin in the sample comprising the serum sample of a subject can then be compared to the average of the relevant population (men, women, or combined population). If the concentration of osteopontin in the subject's serum is more than one standard deviation less than the average, the subject may be deemed to have an on-going or existing CNP formation. Similarly, a subject may be deemed to have an on-going CNP formation if the subject's osteopontin concentration is more than one standard deviation less than the average, or more than two standard deviations less then the average, or more than three standard deviations less than the average, or more than four standard deviations less than the average, or more than five standard deviations less than the average, or more than six standard deviations less than the average. A similar procedure can be used to assess a subject's susceptibility to a CNP formation in other methods disclosed herein.

The invention is also drawn to a method of detecting the presence of calcified nanoparticles in a biological fluid that include separating serous fluid from biological solids in a biological fluid. A first aliquot that includes the serous fluid and cells from a cell line can be made and a second aliquot that includes a complete culture media and cells from the cell line can be made. The first aliquot can be dispensed in a first well and the second aliquot in a second well. The first and second aliquots can be incubated and then the concentration of osteopontin in each of the first aliquot and second aliquot can be determined. An osteopontin factor of the subject can be calculated by dividing the concentration of osteopontin in the first aliquot by the concentration of osteopontin in the second aliquot. The method can indicate the presence of calcified nanoparticles in the biological fluid if the osteopontin factor is greater than about 5.

The cell line can be a monocyte cell line. The cell line can be a monocyte cell line selected from the group consisting of a THP cell line and a Monocytic/Macrophage cell line. The cell line can be any suitable monocytic line, preferably a cell line that is established and readily available.

The concentration of cells from the cell line in the first aliquot can be about the same as the concentration of cells from the cell line in the second aliquot. The concentration of cells from the cell line in the first aliquot can be the same as the concentration of cells from the cell line in the second aliquot. As used herein, “about the same” concentration refers to a difference in concentrations that is less than 15%, less than 5%, less than 2.5% or even less than 1%.

Prior to the addition of the first and second aliquots, the first well and the second well can be free of calcifying nanoparticles. The method can indicate the presence of calcified nanoparticles in the biological fluid if the osteopontin factor is greater than about 10.

Previous means for determining the presence of a CNP formation or susceptibility thereto included enzyme linked immunoassay for the presence of the agents as a qualitative means or cultivating CNPs from an individual and approximating the amount of growth by analyzing the increasing optical density of a culture. Neither of these means assessed the immune response of an individual to the presence of the agents.

It has been unexpectedly discovered that immune cells produce osteopontin in response to the presence of calcified nanoparticles. Measurements of osteopontin production from the immune cells of an individual or from the blood serum/plasma of an individual can be used to assess the immune response. The immune response can be interpreted as that which may ensue if an individual becomes susceptible to ectopic calcification or presents an ongoing response to the presence of calcifying nanoparticles.

In addition, the ratio of the amount of osteopontin produced by immune cells in the presence of CNPs and the amount of osteopontin produced by the same immune cells without the presence of CNPs appears to be an indicator of immune response. As described in more detail in the Examples section, it has been discovered that immune cells from systemic lupus erythrematosus (SLE) patients suffering from excessive production of CNPs are limited to producing approximately 2.2 times more osteopontin when challenged with CNPs in a culture system, whereas the immune cells of healthy individuals produce approximately 22 times more osteopontin when CNPs are present. Thus, it appears that the osteopontin factor of an individual's immune cells, their blood fluids, or both, can be used to determine whether CNPs are present in a fluid, biological or otherwise.

In addition, immune cells from a cell line can be combined with a sample of the fluid to form a first aliquot. A second aliquot, that is free from CNPs, can be formed from immune cells from the cell line and a complete culture media. The osteopontin factor for the fluid can be calculated. The osteopontin factor for the fluid can be compared to a CNP calibration for the cell line that was used. For example, in some cases, if the osteopontin factor is greater than 5, there are calcified nanoparticles present in the sample. Thus it appears that a cell line of the monocytic type can be used as a biological assay of the presence of CNPs. The indicator for a positive assay being the increase in osteopontin released from the cells of the monoytic cell line.

The CNP calibration can be generated by calculating the osteopontin factor for the cell line at a variety of CNP concentrations. The concentration of CNPs used for calculating the CNP calibration can be generated using quantitative techniques for determining the amount of CNPs.

It is to be understood that while the invention has been described in conjunction with the preferred specific embodiments thereof, that the foregoing description as well as the examples which follow are intended to illustrate and not limit the scope of the invention. Other aspects, advantages and modifications within the scope of the invention will be apparent to those skilled in the art to which the invention pertains.

The following Examples are provided as one way to illustrate the invention. It should be understood that the Examples described below are provided for illustrative purposes only and do not in any way define the scope of the invention.

### EXAMPLES

### Materials and Methods

CNP cultures

An exudate sample (100 ul) was diluted into 5 ml of Ultraculture media (Lonza, Rockland Me.) or RPMI media (Sigma, St. Louis) and cultured in a 25 cm² flask, in a humidified incubator, at 37° C. and 5% CO2, for two weeks. Following the incubation period, samples were diluted 1/10 in RPMI 1640, with 10% gamma irradiated, 40 nm filtered fetal bovine serum (FBS) (Hyclone, Logan, Utah) (complete media). Samples from this dilution (1 ml each) were placed in three wells of a 6 well plate (Costar, Sigma, St. Louis Mo.) and a same volume of complete media, without CNPs, was placed in the other three wells. Duplicate plates were incubated at 37° C. for 3 days, until a microscopic biofilm was visible at the bottom of the wells.

CNPs were identified using the 8D10 monoclonal antibody (Nanobac Oy, Kuopio Finland) by a flow cytometry method and by ELISA assay.

Peripheral Blood Mononuclear Cell Purification:

Peripheral blood samples from five SLE patients, and three healthy donors, were obtained by phlebotomy and the mononuclear cell fraction was purified by gradient centrifugation, using Histopaque (Sigma, St Louis Mo.). After two washes, the peripheral blood mononuclear cells (PBMCs) were diluted to 1×10⁶ cell/ml in complete media (RPMI, 10% FBS). The PBMCs were placed in the wells containing CNPs, or complete media alone, at a final volume of 3 ml/well, and incubated at 37° C., 5% CO2, for one week. After the incubation period, non-adherent cells were aspirated and the adherent cells were collected with a rubber scraper (Becton Dickinson, San Jose, Calif.). Adherent and non-adherent cells were centrifuged separately at 400 g for 10 minutes. The supernatant fluid was collected and frozen at −20° C. The cells were re-suspended in complete media, counted in the presence of trypan blue to determine viability, and stained for surface marker expression.

Flow Cytometry Analysis.

Non-adherent PBMCs were stained with the following monoclonal antibodies: anti-CD3FITC and anti-CD19PE (R&D systems, Minneapolis Minn.) following the manufacturer's instructions. Briefly, for each sample, 1×10⁶ cells were centrifuged at 400 g for 10 minutes and the pellets were re-suspended in 100 μl of fluorescence activated cell sorting (FACS) buffer (PBS, 1% BSA, 0.01% sodium azide) containing a polyclonal human IgG (at 1 μg/ml final dilution), to block Fc receptors. Anti-CD19PE and anti-CD3FITC were then added (20 μl of each). Cells were incubated for 30 minutes at approximately room temperature (RT), washed in FACS buffer, re-suspended in 1 ml of FACS buffer and transferred to flow cytometry tubes for analysis. As used herein, room temperature is a temperature within the range from 20 to 24 degrees Celsius. Another sample of non-adherent cells (1×10⁶) was treated with the corresponding isotype controls for PE and FITC (R&D Systems, Minneapolis, Minn.).

Adherent cells (5×10⁵) were centrifuged and resuspended in 100 μl of FACS buffer containing polyclonal human IgG to block Fe receptors, and incubated for 15 minutes at RT. Anti-CD169 monoclonal antibody (Novus Biologicals, Littleton, Colo.) was added, diluted in FACS buffer to a final concentration of 5 μg/ml. For isotype controls, cells were stained with a mouse IgG1 monoclonal antibody (ebiosciences, San Diego, Calif.) (at 5 μg/ml final concentration). The cells were incubated for 1 hr at RT. All the samples were washed twice in FACS buffer and rat anti-mouse IgG1-fluorescein isothiocyanate (FITC) was added (0.5 μg/100 μl ) The cells were incubated for 1 hour at RT and washed twice in FACS buffer. After the second wash, the pellets were resuspended in 100 μl of FACS buffer and anti-CD14APC, anti-CD80 PerCyP, and anti CD86-PE were added (20 μl of each antibody) (Ebiosciences, San Diego, Calif.). Another aliquot of cells was stained with the appropriate isotype controls for APC protein, PerCyP and PE. Cells were incubated for 30 minutes at RT, washed once in FACS buffer, re-suspended in FACS buffer and transferred to flow cytometry tubes for analysis.

Cells were analyzed on a FACSCalibur flow cytometer (BD, San Jose, Calif.), and 15,000 events were collected for each sample. The analysis was performed on Cell Quest software (BD, San Jose, Calif.).

Osteopontin Assay

Supernatant fluids from healthy donors' and SLE patients' PBMCs cultures were thawed and the concentration of osteopontin (OPN) was determined by ELISA (Quantikine OPN ELISA, R&D Systems, Minneapolis, Minn.), according to manufacturer's instructions. Briefly, samples and standards at different concentrations were placed in wells and incubated for 2 hr at RT. The plate was washed with wash buffer provided by the manufacturer and OPN conjugate was added to each well. After incubating for 2 hr at RT, the plate was washed twice, substrate solution was added, the plate incubated for 30 min. and the reaction stopped with stop solution. The plate was read in a microplate, reader set to 450 nm. Another reading at 570 nm was subtracted from the initial readings in order to correct for optical imperfections in the plate. An average of three samples was used to generate a standard curve with Graph Prism V software (San Diego, Calif.), using a four parameter logistic curve fit. This curve was used to reduce the optical density data of the samples to the appropriate concentration, and the dilutions were then accounted for.

Culture of PBMCs with CNPs for Detection of Osteopontin Specific Messenger RNA Synthesis by Microarray Analysis.

CNPs cultures were established in 6 well plates as described above. PBMCs were obtained from SLE and healthy donor peripheral blood samples, and purified by density gradient centrifugation. After washing, cells were re-suspended in RPMI 1640 and 10% defined FBS, with no antibiotics (complete media), at a concentration of 1×10⁶ cells/ml.

Cells (3 ml/well) were placed in the 6 well plates treated with CNPs or complete media, and incubated for seven days at 37° C. After the incubation period, the cells were collected, and centrifuged at 400 g for 10 minutes. The supernatant fluid was frozen at −20° C. and the non-adherent cells were washed twice with cold PBS. The adherent cells were likewise washed twice with cold PBS. The adherent cells incubated with CNPs and those incubated with media alone were lysed with 1 ml/well of TriReagent (Ambion, Austin, Tex.). The lysate was used to further lyse the non-adherent cells that had been pelleted after two washes with PBS. The lysates were transferred to RNAase, DNAase free tubes (Denville Scientific, Plainville N.J.) and frozen at −70° C.

The samples were analyzed using four HEEBO (human exonic evidence-based oligonucleotide) microarrays. HEEBO microarrays were printed by Microarrays Inc. (Nashville, Tenn.) and contained 48,580 70-mer oligonucleotide probes complementary to constitutive exons of most human genes, as well as alternatively spliced exons, ESTs, and control sequences. Total RNA was isolated from the Trizol lysates using Invitrogen's recommended protocol. RNA was further purified by digestion with Rnase-free DNase I (Ambion, Austin, Tex.) for 30 minutes at 37° C. followed by clean-up using RNeasy purification columns (Qiagen, Valencia, Calif.).

Complementary RNA (cRNA) probes were prepared by the method of Van Gelder et al. Briefly, an oligonucleotide containing a 5′-T7-promoter sequence and a 3′ T24VN sequence was used to prime reverse transcription of RNA catalyzed by Superscript II (Invitrogen, Eugene, Oreg.). Double-stranded cDNA was prepared from the 1st strand product by the method of Gubler & Hoffman, and purified on a PCR purification column (Qiagen,Valencia Calif.). The double-stranded cDNA was then used as a template for in vitro transcription with T7 RNA polymerase using a high yield transcription kit (Ambion, Austin, Tex.) and including biotin-16-UTP (Ambion, Austin, Tex.) in the reaction mixture.

Biotinylated cRNA samples were fragmented, diluted in a formamide-containing hybridization buffer, and loaded on to the surface of HEEBO microarray slides enclosed in custom hybridization chambers. The slides were hybridized for 16-18 hours under constant rotation in a Model 400 hybridization oven (Scigene, Sunnyvale, Calif.). After hybridization, the microarray slides were washed under stringent conditions, stained with Streptavidin-Alexa-647 (Invitrogen, Eugene, Oreg.), and scanned using an Axon GenePix 4000B scanner (Molecular Devices, Sunnyvale, Calif.).

### Data Pre-Processing

Spot intensities for each probe were calculated by subtracting median local background from median local foreground for each spot. Spot intensities were transformed by taking the base 2 logarithm of each value. The spot intensities were then normalized by subtracting the 20% trim mean of probes against human constitutive exons and adding back a scaling factor (grand mean of trim means). After removing data for low quality spots, control sequences, and non-human probes, 42,006 human probe intensities remained. The human probes intensities were filtered to identify all probes with intensity above a normalized threshold (log₂ (3* standard deviation of raw local background)+mean of log₂-transformed negative controls), to arrive at 21,646 probes above threshold.

### Differential Expression Analysis

The log2-transformed and normalized spot intensities were examined for differences between stimulated and non-stimulated of>1 log2 unit (2-fold change). National Institute of Ageing Array Analysis software was used to perform hierarchical clustering of the samples and principal component analysis.

### Gene Ontology Analysis

The 21,646 probes above threshold were collapsed to a list of 13,527 Entrez genes. Where multiple probes represented one gene, spot intensities were averaged. The number of genes showing average probe intensity changes of>1 log2 unit between stimulated and control for both lupus and normal cell samples were calculated. Gene ontology categories showing significant over-representation of differentially expressed genes were determined using GenMAPP software (Gladstone Institute). Specifically, the MAPPfinder module of GenMAPP was used to compare the list of Entrez genes showing a probe intensity change of>1 or <−1 log 2 unit between stimulated and control samples to the overall list of 13,527 Entrez genes that were above threshold on the microarray. Significance was determined by permutation of Z scores with correction for multiple comparisons as described in the GenMAPP software manual.

### Results

Effect of CNPs on the Viability of Mononuclear Cells:

PBMCs from SLE patients did not experience any loss of viability after incubation on a microfilm of CNPs, when compared to the viability of PBMCs incubated in media alone.

Similar results were obtained with healthy donor PBMCs incubated with or without CNPs (data not shown).

Effect of CNPs on the Release of Osteopontin from PBMCs.

Osteopontin (OPN), originally named early T-cell activation protein (Eta-1), is produced by macrophages and T-cells when they are activated. OPN production by T cells is dependent on the T-bet transcription factor activation and it activates dendritic cells by up-regulation of co-stimulatory molecules, and by IL-12 and TNF α production. T helper cells, when polarized towards a Th 1 phenotype, secrete OPN in response to IL-12 signaling.

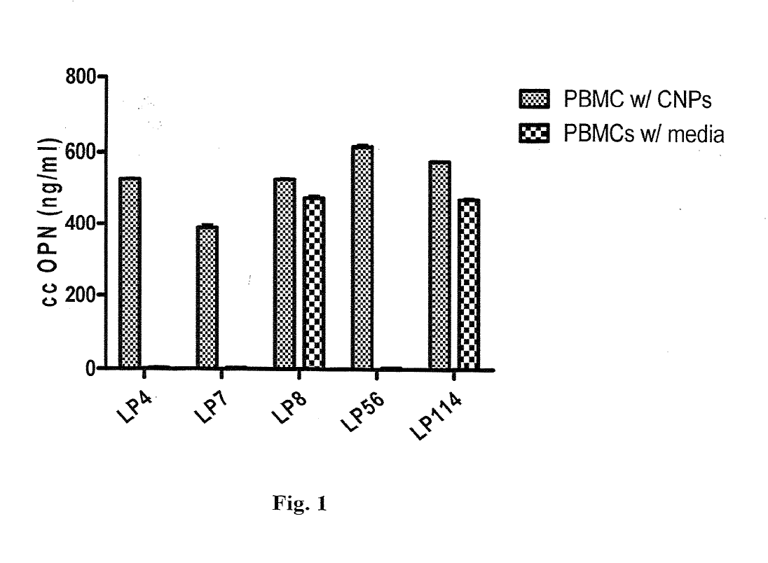

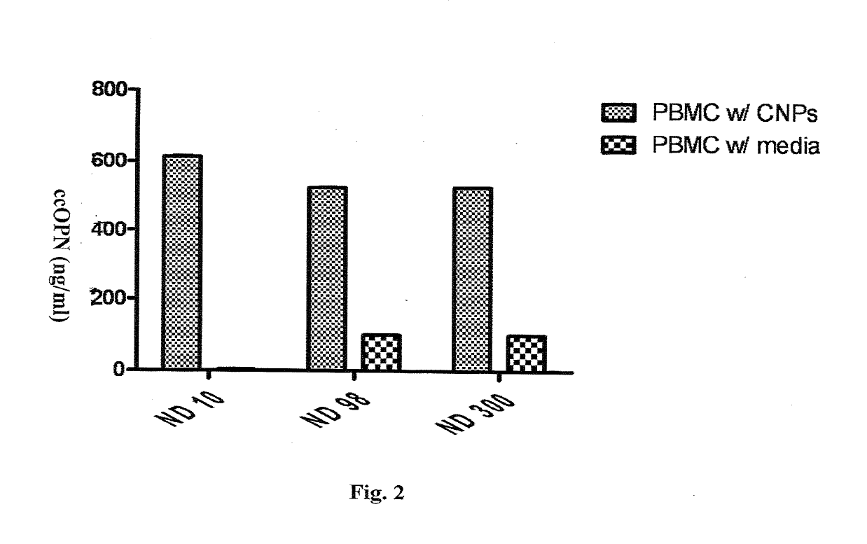

Osteopontin secretion was evaluated in supernatants of PBMCs cultures from both SLE patients and healthy donors, incubated in the presence or absence of CNPs. The concentration of OPN in the supernatants of PBMCs cultured in the presence of CNPs was significantly increased in both cases when compared with the same cultures incubated without CNPs. The results, which are shown in FIGS. 1 and 2, indicate the mean and standard deviation. The assays were done in duplicate with similar results in every case (p<0.05 paired t test). For the data displayed in FIG. 2, the PBMCs were stimulated with CNPs or media alone (p<0.05, paired Student t test).

Microarray Analysis of the Expression of Osteopontin Messenger RNA within PBMCs from Healthy Donor and SLE Patients Incubated in the Presence, or Absence, of CNPs.

It was determined that for a gene to be categorized as down-regulated, the gene must exhibit greater than 50% decrease in expression.

Thirteen hundred sixty-five (1365) probes met the criteria, with 243 probes showing a difference between stimulated and non-stimulated healthy donor and 1199 probes showing differences between stimulated and non-stimulated lupus samples. There were 77 probes showing differences due to stimulation that were common to both healthy and lupus samples. The comparisons were strictly between each donor's cells, incubated or not, with CNPs. When PBMCs from an SU patient were incubated in the presence of CNPs, 765 genes were down-regulated and 434 genes were up-regulated when compared to the gene expression of the patients PBMCs incubated without CNPs. In marked contrast, healthy donor PBMC cultures did not show such an extensive modulation of gene expression (only 109 genes were down-regulated and 134 genes up-regulated) in the presence or absence of CNPs.

The gene for osteopontin was up-regulated twenty two fold in the healthy donor and 2.2 fold in the SLE patient.

### Discussion

It was observed that a human isolate obtained from a lupus panniculitis patient, and sub-cultivated twice in gamma irradiated FBS, did not exhibit marked cytotoxicity toward human peripheral blood mononuclear cells. Previous authors have published that fibroblasts are more susceptible to apoptosis than lymphocytes. Calcifying nanoparticles isolated from bovine serum and rabbit urine have been shown to be markedly cytotoxic to mouse 3T3 fibroblasts. However, the cytotoxic effect observed was attenuated upon subculture of the particles in media alone. The authors concluded that the nanobacteria caused the fibroblasts to become phagocytic, internalize the particles and die. Most noteworthy in their study was the fact that a single human isolate that had been cultivated in gamma irradiated FBS serum was not cytotoxic to mouse fibroblasts.

Remarkably, the co-cultivation of human PBMCs with a well established biofilm of CNPs for one week did not affect the percentages of T and B cells; neither was there a significant up-regulation of the co-stimulatory molecules CD86 and CD80 within the PBMC population. However, CD14 expression showed a shift to lower mean fluorescence intensity which is associated with a monocyte to macrophage transition.

Repeated attempts have been made at microscopic observations of the appearance of enlarged macrophage-like cells following incubation of PBMCs with CNPs. Detsch et al. have shown that hydroxyapatite causes monocytes to form osteoclast-like cells. CNPs are well known to generate crystals of hydroxyapatite. Perhaps the macrophage-like cells that were observed were related to osteoclasts and the transformation of the monocytes was induced by the presence of hydroxyapatite. However, Miller et al. (2004) have shown by electrophoretic analysis that CNPs contain a number of proteins within the particles. Perhaps some of these proteins may show activity towards monocytes as well, but this issue remains unresolved.

In order to elucidate the effects of the CNP isolate on immune cells, a microarray was used to analyze the effects of the particles on gene expression within human PBMCs. The results showed a dramatic twenty two-fold increase in the expression of osteopontin messenger RNA by a healthy donor's PBMCs. PBMCs from a lupus patient whose cells had shown a significant decrease in CD169 also showed a significant but much smaller 2.2 fold increase in osteopontin gene expression when stimulated with CNPs derived from the granulomatous exudate of an SLE patient. OPN ELISA assays confirmed the microarray data. Both SLE patient's and healthy donor's PBMCs secreted significantly higher amounts of OPN when incubated in the presence of CNPs.

Osteopontin (OPN) is a multi-functional molecule, involved in bone remodeling, and in immune responses, specifically cell-mediated and granulomatous inflammation. One important symptom of lupus panniculitis is the presence of inflamed nodules. Lupus panniculitis occurs in 2-3% of patients with systemic lupus erythematosus, most often in association with discoid lesions.

Osteopontin is secreted by T helper cells, macrophages, dendritic cells as well as non-immune cells such as epithelial cells, endothelial cells, smooth muscle cells, fibroblasts, osteoclasts and osteoblasts. Human OPN contains interesting structural domains: an RGD domain, critical for binding integrins, a calcium binding site (residues 202-213), two consensus heparin binding domains (residues 151-160 and 276-283), and residues 86-96 are a hydroxyapatite binding site. The presence of a hydroxyapatite binding site on osteopontin is particularly relevant to the instant findings. The outer surface of CNPs is believed to be covered with hydroxyapatite, and thus the secretion of osteopontin by immune cells in response to their presence may represent a defensive mechanism, perhaps by the macrophage-like cells induced following exposure of CD14 positive monocytes to CNPs. This theory is supported by the finding that OPN has been specifically localized in and around inflammatory cells. Investigators have shown that monocytes rapidly secrete osteopontin when they are stimulated to become macrophages. These authors suggested that osteopontin may recruit and retain macrophages and T-cells at inflamed sites. Osteopontin may also regulate the production of inflammatory cytokines and nitric oxide within macrophages. Thus, CNPs may cause monocytes to transform into macrophages and secrete inflammatory cytokines. The instant findings are consistent with the presence of inflammation and nodular development we and others observe at the sites of lupus associated panniculi.

We claim:

1. A method of measuring the susceptibility of a subject to calcifying nanoparticle formation comprising:
producing a cell culture comprising a complete culture media and peripheral blood mononuclear cells, immune cells, or both, obtained from a peripheral blood mononuclear fraction of a subject;
extracting a first aliquot and a second aliquot from the peripheral blood mononuclear cell culture;
dispensing the first aliquot in a first well and the second aliquot in a second well, wherein a surface of the first well comprises a calcifying nanoparticle biofilm and a surface of the second well is free of calcifying nanoparticles;
incubating the first and second aliquots;
determining the concentration of osteopontin in the first and second aliquots;
calculating an osteopontin factor for the subject; and
comparing the osteopontin factor of the subject to osteopontin factors from other subjects with varying levels of susceptibility to calcifying nanoparticle formation, wherein the osteopontin factor is equal to the concentration of osteopontin in the first aliquot divided by the concentration of osteopontin in the second aliquot.

2. The method of claim 1, wherein the cell culture comprises peripheral blood mononuclear cells from a peripheral blood mononuclear fraction.

3. The method of claim 1, wherein the cell culture comprises immune cells from a peripheral blood mononuclear fraction.

4. The method of claim 1, wherein the culture media is a human blood cell culture that is chemically defined, serum free media, wherein the culture media does not contain any viable nanoparticles capable of replicating.

5. The method of claim 1, wherein the subject is susceptible to calcifying nanoparticle formation if the osteopontin factor is greater than about 5.

6. The method of claim 1, wherein the subject is susceptible to calcifying nanoparticle formation if the osteopontin factor is greater than about 10.

7. A method of measuring the susceptibility of a subject to calcifying nanoparticle formation, comprising:
determining the concentration of osteopontin in a sample comprising serum or defibrinated plasma from a subject; and
comparing the osteopontin concentration of the subject to an average osteopontin concentrations of a population of subjects with a varying, known level of resistance to calcifying nanoparticle formation.

8. The method of claim 7, wherein the population of subjects with a known resistance to calcifying nanoparticle formation are subjects who do not show signs of a calcifying nanoparticle formation, and wherein the susceptibility of a subject to calcifying nanoparticle formation is elevated if the osteopontin concentration of the subject is about 50% or less than the average osteopontin concentration of population of subjects.

9. The method of claim 7, wherein the concentration of osteopontin is determined using ELISA.

10. A method of identifying the presence of an on-going calcifying nanoparticle formation, comprising:
determining the concentration of osteopontin in a sample comprising serum or defibrinated plasma from a subject; and
comparing the osteopontin concentration of the subject to an average osteopontin concentration of a population of subjects with a known resistance to calcifying nanoparticle formation.

11. The method of claim 10, wherein the population of subjects with a known resistance to calcifying nanoparticle formation are subjects who do not show signs of a calcifying nanoparticle formation, and wherein the susceptibility of a subject to calcifying nanoparticle formation is elevated if the osteopontin concentration of the subject is about 50% or less than the average osteopontin concentration of population of subjects.

12. The method of claim 10, wherein the concentration of osteopontin is determined using ELISA.

13. A method of detecting the presence of calcified nanoparticles in a biological fluid, comprising,
separating serous fluid from biological solids in a biological fluid;
mixing a first aliquot comprising the serous fluid and cells from a cell line;
mixing a second aliquot comprising complete culture media and cells from the cell line;
dispensing the first aliquot in a first well and the second aliquot in a second well;
incubating the first and second aliquots;
determining the concentration of osteopontin in the first and second aliquots; and
calculating an osteopontin factor for a subject, wherein the osteopontin factor is equal to the concentration of osteopontin in the first aliquot divided by the concentration of osteopontin in the second aliquot, and wherein calcified nanoparticles are present in the biological fluid if the osteopontin factor is greater than about 5.

14. The method of claim 13, wherein the cell line is a monocyte cell line.

15. The method of claim 13, wherein the cell line is a monocyte cell line selected from the group consisting of a THP cell line and a MonMac cell line.

16. The method of claim 13, wherein a concentration of cells from the cell line in the first aliquot is about the same as the concentration of cells from the cell line in the second aliquot.

17. The method of claim 16, wherein the concentration of cells from the cell line in the first aliquot is the same as the concentration of cells from the cell line in the second aliquot.

18. The method of claim 13, wherein, prior to the addition of the first and second aliquots, the first well and the second well are free of calcifying nanoparticles.

19. The method of claim 13, wherein a calcified nanoparticles are present in the biological fluid if the osteopontin factor is greater than about 10.

20. The method of claim 13, wherein the complete culture media is a human blood cell culture that is generated in serum free media, wherein the culture media does not contain any viable nanoparticles capable of replicating.
